(19) United States
(12) Patent Application Publication
(10) Pub. No.: US 2012/0225849 A1
(43) Pub. Date: Sep. 6, 2012

(54) 2-Methoxyestradiol (2-ME2) Prodrug with Enhanced Bioavailability for Prophylaxis or Treatment of Cancerous or Non-Cancerous Condition

(75) Inventors: Suman KAMBHAMPATI, Leawood, KS (US); Roger A. Rajewski, Lawrence, KS (US); Sushanta K. Banerjee, Lenaxa, KS (US); Mehmet Tanol, Lawrence, KS (US)

(73) Assignees: The University of Kansas, Lawrence, KS (US); U.S. Department of Veterans Affairs, Washington, DC (US)

(21) Appl. No.: 13/408,121

(22) Filed: Feb. 29, 2012

Related U.S. Application Data
(60) Provisional application No. 61/457,327, filed on Mar. 1, 2011.

(51) Int. Cl. A61K 31/661; A61P 19/02; A61P 43/00; C07J 1/00; A61P 35/00

(52) U.S. Cl. 514/120; 552/506

(57) ABSTRACT

A prodrug of 2-methoxyestradiol (2-ME₂) can be used for prophylaxis or treatment of cancer, such as esophageal cancer, prostate cancer, or breast cancer, and/or a non-cancerous condition, such as rheumatoid arthritis or pre-clampsia.

### CROSS-REFERENCE TO RELATED APPLICATIONS

The present application claims priority on prior U.S. Provisional Application Ser. No. 61/457,327, filed Mar. 1, 2011, which is hereby incorporated herein in its entirety by reference.

### STATEMENT REGARDING FEDERALLY SPONSORED RESEARCH OR DEVELOPMENT

The work leading to the present invention was supported by one or more grants from the U.S. Government, and specifically NIH, Centers of Biomedical Research Excellence (COBRE Grant Number P20 RR015563), and Department of Veteran Affairs Merit Review Grant. The U.S. Government therefore has certain rights in the invention.

### FIELD AND BACKGROUND OF THE INVENTION

The present invention is generally directed to cancer prevention and therapy, and more particularly to one or more prodrugs of 2-methoxyestradiol (2-ME₂) that have enhanced solubility and/or bioavailability.

Prostate cancer is one of the most commonly diagnosed cancers in the United States. Among men, prostate cancer is the most common cancer among men of all races and Hispanic origin populations. It is also the leading causes of cancer deaths among men of all races and Hispanic origin populations. It is estimated that nearly 200,000 men are diagnosed and nearly 30,000 will die of prostate cancer annually in the United States. The age-adjusted incidence rate is approximately 159 cases per 100,000 men annually. Additionally, in the United States there were approximately 2.2 million men alive who had a history of prostate cancer (Reference 15).

The global prostate cancer therapy market was estimated at $5.4 billion in 2009. It is one of the largest segments of the oncology market, alongside breast, non-small cell lung and colorectal cancers. The market is forecasted to grow at a compounded annual growth rate of 6 percent to reach $7.8 billion by 2015. This high growth forecast is mainly due to the strong pipeline landscape with innovative first in class drugs and also due to high population growth. Prospective market entrants will face significant challenges including: low treatment seeking rate, low diagnosis rate, low prescription rates and the availability of generics with better efficacy and safety profiles (Reference 16).

Aside from non-melanoma skin cancer, breast cancer is the most common form of cancer in women. Breast cancer is the number one cause of cancer death in Hispanic women and the second most common cause of cancer death in white, black, Asian/Pacific Islander, and American Indian/Alaska Native women. It is estimated that over 191,000 women are diagnosed annually with breast cancer in the United States and over 40,000 women die annually from the disease (Reference 17). The global breast cancer market was estimated at $8.7 billion in 2009 and is forecast to grow at a compounded annual growth rate of 9.6 percent for the next seven years to reach $16.5 billion by 2016. The high projected growth rate is primarily attributable to a strong pipeline. Increases in the treatment seeking population, the diagnosis population and the availability new first-in-class therapies with better safety and efficacy are expected to drive the growth of the breast cancer market (Reference 18).

Rheumatoid arthritis affects an estimated 2.1 million adults in the United States. The disease occurs in all races and ethnic groups but is much more common in women than in men. The global rheumatoid arthritis therapeutics market was valued at $16.8 billion in 2008 and will be driven by the increasing aging population and the steady increase in incidence rates of autoimmune disorders. The market is expected to grow to $26.7 billion with a compounded annual growth rate of 6.8 percent by year 2015. The rheumatoid arthritis market is increasingly becoming more competitive with introduction of novel therapeutics (Reference 19).

In the year 2000, esophageal cancer (EC) was the eighth most common cancer worldwide, with 412,000 new cases, and sixth most common cause for cancer death with 338,000 deaths. In 2002, the number for new cases increased to 462,000, with 386,000 deaths.

There is currently a great demand for developing new treatments for prostate cancer and breast cancer. There are nearly 100 drugs estimated to be in clinical development for prostate cancer. The majority of these are new targeted therapies, including small-molecule tyrosine kinase inhibitors, monoclonal antibodies and therapeutic vaccine candidates. New agents with novel modes of action are also being evaluated in clinical trials, including Dendreon's Provenge, which is likely to be the first therapeutic cancer vaccine to market, and Bristol-Myers Squibb's fully-human monoclonal antibody, ipilimumab. In addition to the development of novel products, some companies are seeking to optimize the life-cycle of drugs already approved in other indications such as Roche's angiogenesis inhibitor, Avastin (bevacizumab), Pfizer's Sutent (sunitinib), Novartis' Gleevec (imatinib), and GlaxoSmithKline's preventative treatment, Avodart (dutasteride), as the most notable examples (Reference 20).

Additionally, there are currently more than 30 marketed products for the treatment of breast cancer, which include chemotherapies, combinations and targeted therapies. Furthermore, the pipeline for breast cancer consists of more than 1,500 molecules currently in development for various disease segments. Approximately 15 percent of the breast cancer pipeline is accounted for by first-in-class molecules (Reference 21).

### ASPECTS OF THE INVENTION

The present disclosure is directed to various aspects of the present invention.

One aspect of the present invention includes a prodrug of an estradiol derivative.

Another aspect of the present invention includes a prodrug of 2-methoxyestradiol (2-ME₂).

Another aspect of the present invention includes a novel chemotherapy agent which could inhibit tumor cell growth and/or proliferation without any of the usual chemotherapy-induced side effects.

Another aspect of the present invention includes a novel chemotherapy and/or a chemopreventive agent which has enhanced bioavailability, aqueous solubility, and/or bio-efficacy.

Another aspect of the present invention includes a prodrug of 2-ME₂, which has enhanced bioavailability than the native 2-ME₂. The prodrug of 2-ME₂ would be metabolized in vivo to release its active metabolite 2-ME₂, which would increase the selectivity of 2-ME₂ for an intended tumor target and improve its anticancer potential and/or properties.

Another aspect of the present invention includes a prodrug of 2-ME₂, which can be used for prophylaxis or treatment of esophageal cancer, prostate cancer, breast cancer, rheumatoid arthritis, and/or pre-clampsia.

Another aspect of the present invention includes a compound having the following general structure:

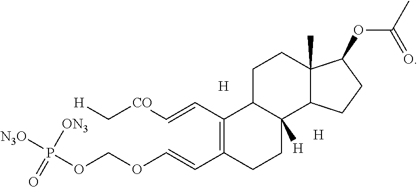

Another aspect of the present invention includes a compound having the general formula C₂₂H₂₉Na₂O₈P.

Another aspect of the present invention includes a chemotherapy agent for prophylaxis or treatment of cancer, or a non-cancerous condition, including a compound having the following general structure:

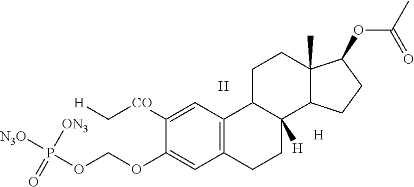

Another aspect of the present invention includes a chemotherapy agent for prophylaxis or treatment of cancer, or a non-cancerous condition, including a compound having the general formula C₂₂H₂₉Na₂O₈P.

Another aspect of the present invention includes a prodrug including 2-methoxyestradiol (2-ME₂) with a hydrophilic moiety at the 3-position.

Another aspect of the present invention includes a prodrug including 2-methoxyestradiol (2-ME₂) with an ester moiety at the 17-position.

Another aspect of the present invention includes a prodrug including 2-methoxyestradiol (2-ME₂) with a bioreversible hydrophilic moiety at the 3-position and an ester moiety at the 17-position.

Another aspect of the present invention includes a method of enhancing bio-efficacy and/or bioavailability of 2-methoxyestradiol (2-ME₂) in a living being, which includes: providing a prodrug including 2-methoxyestradiol (2-ME₂) with a bioreversible hydrophilic moiety at the 3-position and an ester moiety at the 17-position, administering the prodrug to the living being, cleaving off the hydrophilic moiety from the 3-position pre-systemically and/or systemically, and masking the 17-position during a first-pass through the intestinal epithelium and liver.

Another aspect of the present invention includes a method for prophylaxis or treatment of cancer, or a non-cancerous condition, which includes administering a predetermined dose of a medicinal agent to a living being in need thereof, wherein the medicinal agent includes a prodrug of 2-methoxyestradiol (2-ME₂).

In summary, the present invention is directed to an improvement over an existing drug, 2-methoxyestradiol (2-ME₂). It involves the design of one or more prodrugs of 2-ME₂ to overcome the poor bioavailability associated with native 2-ME₂. The prodrug version(s) of 2-ME₂ (henceforth referred to as Pro-2ME₂ or 2ME2-PD) would preferably be metabolized in vivo to release its active metabolite-2-ME₂, which would thereby increase the selectivity of the 2-ME₂ for its intended tumor target and ultimately improve its anticancer potential.

### BRIEF DESCRIPTION OF THE DRAWINGS

One of the above and other aspects, novel features and advantages of the present invention will become apparent from the following detailed description of the non-limiting preferred embodiment(s) of invention, illustrated in the accompanying drawings, wherein:

FIG. 1 illustrates plasma pharmacokinetics of 2-ME₂ (native 2-methoxyestradiol) following intravenous (iv) and oral administration of native 2-ME₂ (native) or 2-ME₂-PD (prodrug); and

FIG. 2 illustrates in vivo anti-tumor effects of 2-ME₂ prodrug.

### DETAILED DESCRIPTION OF THE PREFERRED EMBODIMENT(S) OF THE INVENTION

Estrogens occurring naturally in the body are metabolized to catecholestrogens (2- and 4-hydroxyestradiol) by the cytochrome P450 enzymes. 2-Hydroxy catecholestrogens are further metabolized by catechol-O-methyltransferase to 2-methoxyestradiol (2-ME₂ or 2-ME2), which is known to be protective against tumor formation (Reference 13). 2-Methoxyestradiol exhibits potent apoptotic activity against rapidly growing tumor cells and inhibits angiogenesis by reducing endothelial cell proliferation and inducing endothelial cell apoptosis. This agent also inhibits tumor cell growth by binding to tubulin, resulting in antimitotic activity, and by inducing caspase activation, resulting in cell cycle arrest in the G2 phase, DNA fragmentation, and apoptosis (Reference 14). The exact mechanism of action of 2-ME₂ is still unclear, but it has been shown to be effective in preventing tumor growth in a variety of cell lines.

The present invention is an improvement of the existing drug, 2-ME₂. It involves the design of one or more prodrugs of 2-ME₂ to overcome the poor bioavailability of native 2-ME₂. The prodrug is directed at increasing aqueous solubility/dissolution rates through addition of a 1) bioreversible hydrophilic group at the 3-position of the molecule, and altering metabolism by masking the 17-position through 2) covalent addition of an ester moiety. The 3-position promoiety is designed to preferably be cleaved pre-systemically at the brush-border of the intestinal epithelium providing high local concentrations of the prodrug intermediate for intestinal absorption. (The 3-position promoiety may additionally or alternatively be cleaved-off systemically.) On the first-pass through the intestinal epithelium and liver, the 17-position will be masked and undergoing de-esterification. The design of the prodrug will result in increased systemic exposure to 2-ME₂.

2-Methoxyestradiol (2-ME₂) is an estradiol derivative that acts as a microtubule destabilizing agent at pharmacological doses (References 1 and 2). Recent data suggests that 2-ME₂ is effective against different tumor subtypes and has demonstrated potent antiproliferative and pro-apoptotic properties both in vitro and in vivo settings (References 1-7). Encouraged by the preclinical experience, 2-ME₂ was tested in phase I studies involving patients with solid tumors (Reference 8) and breast cancer (Reference 9) and, in a phase II setting, in prostate cancer patients (Reference 10). One of the consistent end-points in all three studies included pharmacokinetic testing of 2-ME₂, when administered orally. Generally, irrespective of the tumor types tested, in the majority of the patients, large interpatient and intrapatient variability of 2-ME₂ pharmacokinetics was reported, which was ascribed to the poor bioavailability of 2-ME₂ (References 8-10).

Despite the high level of clinical research activity with 2-ME₂, the reasons for poor bioavailability and low systemic concentrations of 2-ME₂ observed after oral dosing, even at very high doses in patients, are not well understood. The major barriers to poor oral drug delivery and systemic exposure of 2-ME₂ include, but are not limited to, formulation, solubility, permeability, transporter effect, and first-pass metabolism. These are summarized below.

### Formulation

2-ME₂ is formulated as 200 mg capsules with lactose, sodium starch glycolate, colloidal silicon dioxide, and magnesium stearate (Panzem®, Entremed Inc.). Due to the limited aqueous solubility of 2-ME₂, extremely high doses of this formulation have been given clinically in an attempt to attain systemically useful 2-ME₂ plasma levels (References 8-10). In all these studies, the AUC (area under curve) for systemic exposure to 2-ME₂ did not correlate with dose. There was no significant increase in exposure with increasing dose. Because of these clinical challenges, Entremed is pursuing a nanocrystalline formulation of 2-ME₂, Panzem NCD® (United States). Although initial studies suggest some improvement in oral bioavailability, results indicate there is still significant interpatient variability of 2-ME₂ pharmacokinetics with this formulation and the required oral dose needed to reach adequate systemic concentrations is still substantial (Reference 11). In addition, in a phase II study in prostate cancer, in which the daily oral dose was 6000 mg, there was significant gastrointestinal toxicity, raising concerns about the GI tolerability of this particular formulation (Reference 12).

### Solubility

2-ME₂ is a poorly soluble compound with a predicted aqueous solubility of 4.8 micrograms/mL (Calculated using Advanced Chemistry Development Software V8.14 for Solaris). 2-ME₂ possesses poor aqueous solubility spanning the complete pH profile of the gastrointestinal tract. Since bioavailability has been increased with the nanocrystal formulation (10-K SEC Filing, filed by Entremed Inc. on Mar. 6, 2008), there is evidence that a more soluble form of 2-ME₂ may help increase its absolute bioavailability.

### Permeability

The predicted log P of 2-ME₂ is 3.84 (Calculated using Advanced Chemistry Development Software V8.14 for Solaris). It is expected that a high fraction of soluble 2-ME₂ will permeate from the apical to the basolateral side of the GI epithelial lining and get through to the portal circulation. Permeability of 2-ME₂ is thus not believed to be a major factor affecting the bioavailability.

### Transporters

The poor solubility of 2-ME₂ can theoretically limit the concentrations entering the enterocytes, thereby preventing the saturation of drug transporters. This phenomenon remains to be further studied.

### First-Pass Metabolism

The first-pass metabolism of an orally administered drug usually occurs within the gastrointestinal (GI) epithelium and liver. In both animal and human studies, only a small fraction of an orally administered 2-ME₂ dose (less than 0.1%) and its metabolites (less than 1%) are recovered in the urine (Reference 13). Of the 2-ME₂ that reaches the urine, the major metabolite is the glucuronide. The low recovery of 2-ME₂ and metabolites in the urine suggests that 2-ME₂ is not a high first-pass clearance drug. This is supported by the observed increase in absolute bioavailability observed with the Panzem NCD® formulation.

Clinical studies in humans and in vivo studies in rodents have shown that orally absorbed 2-ME₂ is metabolized by oxidation at the 17 position (2-methoxyestrone) and phase II glucuronidation at positions 3 and 17 with clearance through the kidney (References 3 and 13). The conjugated forms of 2-ME₂ are inactive, and oxidation to 2-methoxyestrone results in 10-to 100-fold loss in activity in vitro (References 3, 13 and 19). However, approximately only 1% of an orally administered dose of 2-ME₂ is recovered in the urine suggesting both solubility and metabolism as barriers to systemic delivery (Reference 13).

To overcome those barriers, we have designed a prodrug of 2-ME₂ directed at (i) increasing aqueous solubility/dissolution rate through addition of a bioreversible hydrophilic group at the 3-position, and (ii) altering metabolism by masking the 17-position through covalent addition of an ester moiety. The 3-position promoiety is designed to preferably be cleaved pre-systemically at or adjacent the brush-border of the intestinal epithelium providing high local concentrations of the prodrug intermediate for intestinal absorption. (The 3-position promoiety may additionally or alternatively be cleaved off systemically.) On the first-pass through the intestinal epithelium and liver, the 17-position will be masked and undergoing de-esterification. The result will be increased systemic exposure to 2-ME₂.

### Methods and Results

Generic 2-ME₂ Prodrug Synthesis Procedure

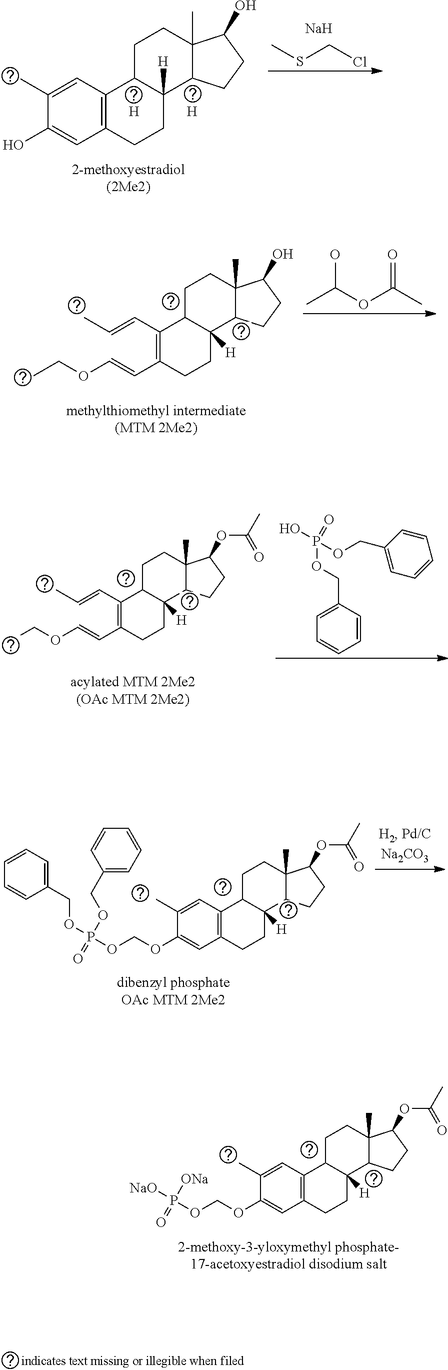

The following steps illustrate the synthesis of main compound of the present invention—disodium(((8R,9S,13S,14S,17S)-17-acetoxy-2-methoxy-13-methyl-7,8,9,11,12,13,14,15,16,17-decahydro-6H-cyclopenta[a]phenanthren-3-yl)oxy)methyl phosphate (C₂₂H₂₉Na₂O₈P).

### Step 1

300 mg (8R,9S,13S,14S,17S)-2-methoxy-13-methyl-7,8,9,11,12,13,14,15,16,17-decahydro-6H-cyclopenta[a]phenanthren-17-ol (2-methoxy estradiol; 0.1 mmol) and 0.140 mL (chloromethyl)(methyl)sulfane (1.67 mmol) were dissolved in 20-mL of dry dimethylformamide. 150 mg of 60% sodium hydride was added and the mixture was stirred at room temperature for one hour. After this time the solvent was removed in vacuo and the resulting solids dissolved in ethyl acetate. The organic layer was washed with water then filtered through silica gel. The ethyl acetate was removed in vacuo. The solids were dissolved in a 1:2 v/v mixture of ethyl acetate and hexanes and the major product isolated with elution on a silica gel column with the same solvents (R_{f}=0.45) to provide 313 mg (8R,9S,13S,14S,17S)-2-methoxy-13-methyl-3-((methylthio)methoxy)-7,8,9,11,12,13,14,15,16,17-decahydro-6H-cyclopenta[a]phenanthren-17-ol (86% yield).

### Step 2

208 mg (8R,9S,13S,14S,17S)-2-methoxy-13-methyl-3-((methylthio)methoxy)-7,8,9,11,12,13,14,15,16,17-decahydro-6H-cyclopenta[a]phenanthren-17-ol (0.57 mmol) and 126 mg acetic anhydride (1.23 mmol, 3 eqv.) were dissolved in 10-mL of dry pyridine at 0° C. The reaction was stirred overnight and allowed to come to room temperature. The solvent was removed in vacuo and the resulting solid was dissolved in a 1:2 v/v mixture of ethyl acetate and hexanes and the major product isolated with elution on a silica gel column with the same solvents (R_{f}=0.81) to provide 157 mg (8R,9S,13S,14S,17S)-2-methoxy-13-methyl-3-((methylthio)methoxy)-7,8,9,11,12,13,14,15,16,17-decahydro-6H-cyclopenta[a]phenanthren-17-yl acetate (68% yield).

### Step 3

157 mg (8R,9S,13S,14S,17S)-2-methoxy-13-methyl-3-((methylthio)methoxy)-7,8,9,11,12,13,14,15,16,17-decahydro-6H-cyclopenta[a]phenanthren-17-yl acetate (0.39 mmol), 325 mg dibenzyl hydrogen phosphate (1.16 mmol, 3 eqv.), and 320 mg N-iodosuccinimide (1.42 mmol, 3.6 eqv.) were dissolved in 5-mL of dry tetrahydrofuran at room temperature. The reaction was stirred one hour after which time the solvent was removed in vacuo and the resulting solid was dissolved in a 1:2 v/v mixture of ethyl acetate and hexanes and the major product isolated with elution on a silica gel column with gradient elution (1:2 v/v ethyl acetate:hexanes to 1:1 v/v ethyl acetate:hexanes; R_{f}=0.62) to provide 120 mg (8R,9S,13S,14S,17S)-3-(((bis(benzyloxy)phosphoryl)oxy)methoxy)-2-methoxy-13-methyl-7,8,9,11,12,13,14,15,16,17-decahydro-6H-cyclopenta[a]phenanthren-17-yl acetate (49% yield).

### Step 4

50 mg (8R,9S,13S,14S,17S)-3-(((bis(benzyloxy)phosphoryl)oxy)methoxy)-2-methoxy-13-methyl-7,8,9,11,12,13,14,15,16,17-decahydro-6H-cyclopenta[a]phenanthren-17-yl acetate (0.079 mmol) was dissolved in a mixture of 2 mL water and 25 mL tetrohydrofuran at room temperature. 11 mg disodium carbonate monohydrate and 50 mg 10% palladium on carbon were added and the reaction was stirred two hours under hydrogen at atmospheric pressure. The mixture was then filtered through a 0.45 micron Nylon filter and lyophilized to provide 39 mg of the title compound (100% yield). Identity was confirmed by mass spectroscopy using a Shimadzu 2010 single quadrupole spectrometer in negative ion mode (free acid theoretical mass: 454.45 amu, found 452.95 amu).

It is noted that alkyl anhydride in Step 2 may be replaced by other straight chain, branched chain, and cyclic alkyl anhydrides to produce the analogues of the compound of the present invention with differing position 17 esters. These analogues will have differing rates of esterase cleavage and may provide greater metabolic protection compared to the main compound of the present invention.

### In vivo Evaluation of Disodium(((8R,9S,13S,14S,17S)-17-acetoxy-2-methoxy-13-methyl-7,8,9,11,12,13,14,15,16,17-decahydro-6H-cyclopenta[a]phenanthren-3-yl)oxy)methyl phosphate in Rats

The main compound of the present invention (2ME2-PD) and the native compound (8R,9S,13S,14S,17S)-2-methoxy-13-methyl-7,8,9,11,12,13,14,15,16,17-decahydro-6H-cyclopenta[a]phenanthren-17-ol (2-ME₂) were dissolved independently in 0.1 M Captisol® at concentrations between 10 mg/mL and 21 mg/mL. The solutions were sterile filtered through 0.2 micron filters prior to use.

Animal studies were approved and conducted in accordance with the guidelines of the Institutional Animal Care and Use Committee. A cannulated rat model was used to study the intravenous (iv) and oral absorption 2-ME₂ and 2-ME₂-PD.

Sprague Dawley rats (male, 250-300 gram, Charles River Laboratories) were implanted with carotid artery, and jugular and/or femoral vein catheters. These studies were automated with the animals connected to the Culex Automated Pharmacology System allowing for direct comparison of pharmacokinetic behavior between the orally and iv administered compounds in the same animal. Following surgery, the animals were connected to the Culex and allowed to recover and acclimate. Fasted animals were dosed intravenously and orally with solutions of 2-ME₂-PD in 0.1 M Captisol® (CyDex Pharmaceuticals, Inc) and 2-ME₂ parent in 0.2 M HP-13-CD (Sigma-Aldrich). The dose levels are provided in Table I (below).

Oral doses were given via gavage to the animal under light anesthesia. Blood was sampled at times ranging from five to 1440 minutes into heparinized vials stored on the chilled fraction collector, and remained there until sampling was complete. Blood samples were processed to plasma via centrifugation and stored at −80° C. until analysis for 2-ME₂-PD, 2-ME₂ and associated metabolite concentration by liquid chromatography-mass spectrometry (LC-MS/MS). Bioanalytical methods for the 2-ME₂-PD and 2-ME₂ analysis were modified from those of Lakhani et. al. Quantitation was made relative to deuterated internal standards.

TABLE I
Rat 2-ME₂ and 2-ME₂-PD Dosing Summary

| | | | | Number of |
|---|---|---|---|---|
| Dose Route | Dose Level | | Compound | Animals |
| Intravenous | 5.21 | mg/kg | 2-ME₂ | 1 |
| Oral | 5.21 | mg/kg | 2-ME₂ | 2 |
| Intravenous | 10 | mg/kg | 2-ME₂-PD | 1 |
| Oral | 20 | mg/kg | 2-ME₂-PD | 2 |
| Oral | 31.5 | mg/kg | 2-ME₂-PD | 1 |

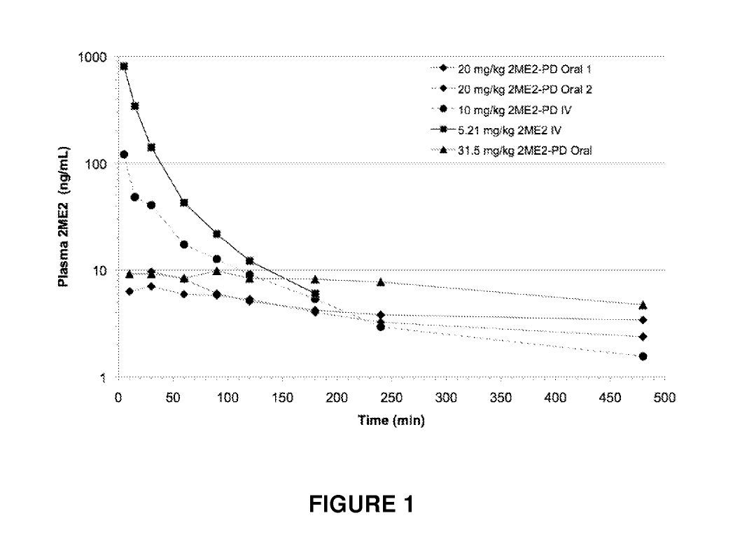

Pharmacokinetic analysis of the resulting plasma concentration time data was performed using PK Solution software (Summit PK). Results for the studies conducted are illustrated in FIG. 1 and Table II (below). For oral doses of 2-ME₂, no compound was found in plasma at all time points studied (<2 ng/mL LOQ). All samples from times greater than 480 minutes had 2-ME₂ levels that were below the limit of quantitation.

The data suggest that the 2-ME₂ prodrug (2-ME₂-PD) strategy employed enables the delivery of 2-ME₂ to the systemic circulation. The approach provides absolute bioavailabilities from oral administration of the 2-ME₂ prodrug in the 4-5% range (0-480 minutes) in these limited studies.

TABLE II
Summary of 2-ME₂ and 2-ME₂-PD Pharmacokinetic Parameters

| | 2-ME₂ | 2-ME₂-PD | | |
|---|---|---|---|---|
| Route | Intravenous | Intravenous | Oral² | Oral |
| Dose (mg/kg) | 5.21 | 10 | 20 | 31.5 |
| Corrected Dose | 5.21 | 6.06 | 12.13 | 19.11 |
| (mg/kg) | | | | |
| AUCₜ₋₄₈₀ | 19253 | 4967 | 2006 | 3516 |
| (ng · min · mL⁻¹) | | | | |
| Cₘₐₓ (ng/mL) | 1221 | 121 | 8.4 | 9.9 |
| T_{1/2} (min) | 59 | 262 | 807 | 411 |
| Bioavailability¹ (%) | 100 | 21.8 | 4.4 | 4.9 |

¹Relative to 2ME₂ iv and corrected for dose; AUC through 480 minutes
²Average of two animals

### Pro-2-ME₂ (2-ME₂-PD) Inhibits the Growth of Barrett's Esophageal Adenocarcinoma (BEAC) Xenografts

After demonstrating the in vitro antitumor properties of 2-ME₂ against OE33 growth and invasion, we determined the in vivo effects of 2-ME₂ on OE33-generated xenografts. We injected ˜2.0×10⁶ OE33 cells subcutaneously into the left hind leg flank of each nude mice (n=2) for the development of tumor. The mice were divided into two groups (two mice per group) with a control group and Pro-2-ME₂ treatment group. After forming palpable tumors, the nude mice bearing xenografts of OE33 cells were given daily pro-2-ME₂ doses (75 mg/kg/day) by orogastric feeding or vehicle (control). Pro-2-ME₂ was dissolved in 500 μl of saline water. We used 500 μl of saline water as a vehicle control. Tumor growth was monitored for 8 days by measuring two perpendicular diameters twice weekly. Tumor volume was calculated according to the formula V=a×b²/2, where a and b are the largest and smallest diameters, respectively.

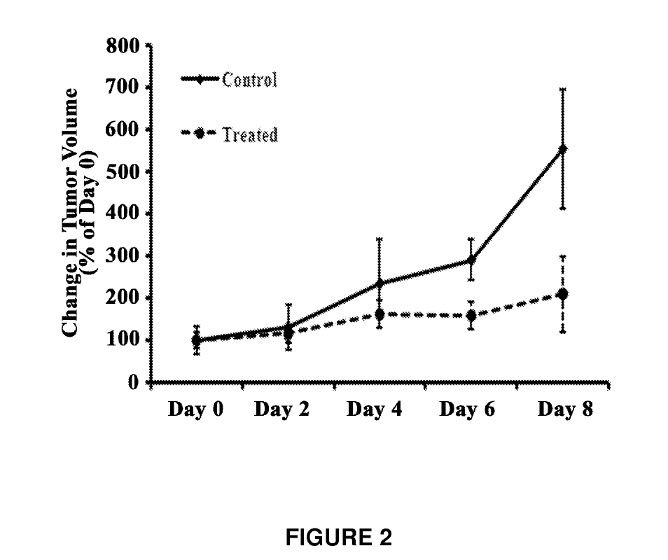

As illustrated in FIG. 2, our data shows that pro-2-ME₂ (2-ME₂-PD) could significantly inhibit the growth of OE33 tumor implants in nude mice, compared to vehicular control fed in vivo OE33 implants.

### CONCLUSION

The results obtained indicate that the growth of OE33 xenografts was significantly inhibited in the pro-2-ME₂ group, than in animals treated with vehicle (control).

While this invention has been described as having preferred sequences, ranges, steps, materials, structures, components, features, and/or designs, it is understood that it is capable of further modifications, uses, and/or adaptations of the invention following in general the principle of the invention, and including such departures from the present disclosure as those come within the known or customary practice in the art to which the invention pertains, and as may be applied to the central features herein before set forth, and fall within the scope of the invention and of the limits of the appended claims.

### REFERENCES

The following references, and those cited in the disclosure herein, are hereby incorporated herein in their entirety by reference.
(1) Mooberry S L. New insights into 2-methoxyestradiol, a promising antiangiogenic and antitumor agent. Curr Opin Oncol 2003 November; 15(6):425-30.
(2) Klauber N, Parangi S, Flynn E, Hamel E, D'Amato R J. Inhibition of angiogenesis and breast cancer in mice by the microtubule inhibitors 2-methoxyestradiol and taxol. Cancer Res 1997 Jan. 1; 57(1):81-6.
(3) Fotsis T, Zhang Y, Pepper M S, Adlercreutz H, Montesano R, Nawroth P P, et al. The endogenous oestrogen metabolite 2-methoxyoestradiol inhibits angiogenesis and suppresses tumour growth. Nature 1994 Mar. 17; 368(6468):237-9.
(4) Zoubine M N, Weston A P, Johnson D C, Campbell D R, Banerjee S K. 2-methoxyestradiol-induced growth suppression and lethality in estrogen-responsive MCF-7 cells may be mediated by down regulation of p34cdc2 and cyclin B1 expression. Int J Oncol 1999 October; 15(4):639-46.
(5) Banerjeei S K, Zoubine M N, Sarkar D K, Weston A P, Shah J H, Campbell D R. 2-Methoxyestradiol blocks estrogen-induced rat pituitary tumor growth and tumor angiogenesis: possible role of vascular endothelial growth factor. Anticancer Res 2000 July; 20(4):2641-5.
(6) Banerjee S N, Sengupta K, Banerjee S, Saxena N K, Banerjee S K. 2-Methoxyestradiol exhibits a biphasic effect on VEGF-A in tumor cells and upregulation is mediated through ER-alpha: a possible signaling pathway associated with the impact of 2-ME2 on proliferative cells. Neoplasia 2003 September; 5(5):417-26.
(7) Kambhampati S, Banerjee S, Dhar K, Mehta S, Hague I, Dhar G, et al. 2-methoxyestradiol inhibits Barrett's esophageal adenocarcinoma growth and differentiation through differential regulation of the beta-catenin-E-cadherin axis. Mol Cancer Ther. 2010 March; 9(3):523-34. Epub 2010 Mar. 2.
(8) Dahut W L, Lakhani N J, Gulley J L, Arlen P M, Kohn E C, Kotz H, et al. Phase I clinical trial of oral 2-methoxyestradiol, an antiangiogenic and apoptotic agent, in patients with solid tumors. Cancer Biol Ther 2006 January; 5(1):22-7.
(9) James J, Murry D J, Treston A M, Storniolo A M, Sledge G W, Sidor C, et al. Phase I safety, pharmacokinetic and pharmacodynamic studies of 2-methoxyestradiol alone or in combination with docetaxel in patients with locally recurrent or metastatic breast cancer. Invest New Drugs 2007 February; 25(1):41-8.
(10) Sweeney C, Liu G, Yiannoutsos C, Kolesar J, Horvath D, Staab M J, et al. A phase II multicenter, randomized, double-blind, safety trial assessing the pharmacokinetics, pharmacodynamics, and efficacy of oral 2-methoxyestradiol capsules in hormone-refractory prostate cancer. Clin Cancer Res 2005 Sep. 15; 11(18):6625-33.
(11) Tevaarwerk A J, Holen K D, Alberti D B, Sidor C, Arnott J, Quon C, et al. Phase I trial of 2-methoxyestradiol NanoCrystal dispersion in advanced solid malignancies. Clin Cancer Res. 2009 Feb. 15; 15(4):1460-5.
(12) Harrison M R, Hahn N M, Pili R, Oh W K, Hammers H, Sweeney C, et al. A phase II study of 2-methoxyestradiol (2ME2) NanoCrystal(R) dispersion (NCD) in patients with taxane-refractory, metastatic castrate-resistant prostate cancer (CRPC). Invest New Drugs. 2010 May 25. [Epub ahead of print]
(13) 2-Methoxyestradiol, A Promising Anticancer Agent, Pharmacotherapy, Mar. 17, 2003.
(14) National Cancer Institute, www.cancer.gov.
(15) National Cancer Institute, Surveillance Epidemiology and End Results, 2009.
(16) Prostate Cancer—Drug Pipeline Analysis and Market Forecasts to 2015, GlobalData, Jan. 7, 2010.
(17) National Program of Cancer Registries, Centers for Disease Control and Prevention.
(18) Breast Cancer—Drug Pipeline Analysis and Market Forecasts to 2016, GlobalData, Jan. 27, 2010.
(19) Rheumatoid Arthritis—Drug Pipeline Analysis and Market Forecasts to 2015, GlobalData, Jan. 7, 2010.
(20) Cancer Drug Discoveries: What the Future Holds, Prostate Cancer Chapter, Espicom Business Intelligence, November, 2009.
(21) Breast Cancer—Drug Pipeline Analysis and Market Forecasts to 2016, GlobalData, Jan. 27, 2010.
(22) Ray G, Dhar G, Van Veldhuizen P J, Banerjee S, Saxena N K, Sengupta K, et al. Modulation of cell-cycle regulatory signaling network by 2-methoxyestradiol in prostate cancer cells is mediated through multiple signal transduction pathways. Biochemistry 2006 Mar. 21; 45(11):3703-13.
(23) Van Veldhuizen P J, Ray G, Banerjee S, Dhar G, Kambhampati S, Dhar A, et al. 2-Methoxyestradiol modulates beta-catenin in prostate cancer cells: a possible mediator of 2-methoxyestradiol-induced inhibition of cell growth. Int J Cancer 2008 Feb. 1; 122(3):567-71.
(24) Liu G, Quon C Y, Sidor C, Feierabend C, Eun J, et al. Phase I trial of 2-methoxyestradiol (2ME2), administered orally as a Nanocrystal® colloidal dispersion (NCD), in patients with advanced cancer. Clin Cancer Res 2005 December: 11(24 Suppl): 9035s.
(25) Harrison M R, Hahn N, Pili R, Oh W K, Kim K, et al. Phase II study of 2-methoxyestradiol (2ME2) Nanocrystal® dispersion (NCD) in patients with taxane-refractory, metastatic hormone-refractory prostate cancer (HRPC). J Clin Oncol 2008 May; 26.
(26) Lakhani N J, Sparreboom A, Xu X, Veenstra T D, Venitz J, Dahut W L, et al. Characterization of in vitro and in vivo metabolic pathways of the investigational anticancer agent, 2-methoxyestradiol. J Pharm Sci 2007 July; 96(7):1821-31.
(27) Parkin D M, Lancet Oncol 2001; 2: 533-543.
(28) Parkin D M, C A Cancer J Clin. 2005; 55:74-108.
(29) Jemal A, C A Cancer J Clin. 2007.
(30) Pohl, H. et al. J Natl Cancer Inst 2005; 97:142-146.
(31) Boulton-Jones et al, Helicobacter 1999; 4: 281.
(32) Vaughan et al, Cancer Epidemiol. Biomarkers Prev. 2002; 11: 745.
(33) Rudolph et al, J Natl. Cancer Inst. 2003: 95: 750.
(34) Brown et al, Surg. Oncol. Clin. N. Am. 2002; 11: 235
(35) Corley et al, Gastroenterol. 2003; 124: 47.
(36) Wild et al 2003 Nat Rev Cancer.
(37) Klauber et al, Cancer Res. 1997; 57: 81.
(38) Fotsis et al, Nature 1994; 368: 237.
(39) Davoodpour et al, J. Biol. Chem. 2005; 280: 14773.
(40) Dingli et al, Clin. Cancer Res. 2002; 8: 3948.
(41) Banerjee et al, Neoplasia 2003; 5: 417.
(42) Ray et al, Biochemistry 2006; 45: 3703.
(43) Vanvelduizen et al, Int J Cancer. 2008; 122: 567.
(44) Kambhampati et al, Molecular Cancer Therapeutics (In press).
(45) Lakhani N J et al, Rapid Commun Mass Spectrom 2005; 19:1176.
(46) Laknani N J et al, J Pharm Sci 2007; 96:1821.
(47) Dahut W L et al, Cancer Biol Ther 2006; 5:22.
(48) James J et al, Invest New Drugs 2007; 25:41.
(49) Sweeney C et al, Clin Cancer Res 2005; 11: 6625.

What is claimed is:

1. A compound comprising the following general structure:

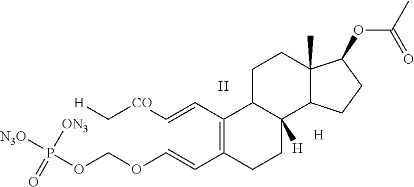

2. The compound of claim 1, wherein the compound comprises a prodrug of an estradiol derivative.

3. The compound of claim 2, wherein the estradiol derivative comprises 2-methoxyestradiol (2-ME₂).

4. A metabolite comprising the compound of claim 1.

5. A metabolite of the compound of claim 1.

6. A composition comprising the compound of claim 1.

7. A pharmaceutical formulation or composition comprising the compound of claim 1.

8. A compound comprising the general formula C₂₂H₂₉Na₂O₈P.

9. The compound of claim 8, wherein the compound comprises a prodrug of an estradiol derivative.

10. The compound of claim 9, wherein the estradiol derivative comprises 2-methoxyestradiol (2-ME₂).

11. A metabolite comprising the compound of claim 8.

12. A metabolite of the compound of claim 8.

13. A composition comprising the compound of claim 8.

14. A pharmaceutical formulation or composition comprising the compound of claim 8.

15. A chemotherapy agent for prophylaxis or treatment of cancer, or a non-cancerous condition, comprising a compound having the following general structure:

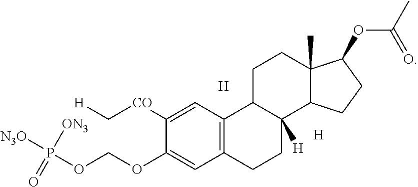

16. The chemotherapy agent of claim 15, wherein the cancer comprises at least one condition or disorder selected from the group consisting of esophageal cancer, prostate cancer, and breast cancer.

17. The chemotherapy agent of claim 15, wherein the non-cancerous condition comprises rheumatoid arthritis, pre-clampsia, or both.

18. The compound of claim 15, wherein the compound comprises a prodrug of an estradiol derivative.

19. The compound of claim 18, wherein the estradiol derivative comprises 2-methoxyestradiol (2-ME₂).

20. A metabolite comprising the compound of claim 15.

21. A metabolite of the compound of claim 15.

22. A composition comprising the compound of claim 15.

23. A pharmaceutical formulation or composition comprising the compound of claim 15.

24. A chemotherapy agent for prophylaxis or treatment of cancer, or a non-cancerous condition, comprising a compound having the general formula C₂₂H₂₉Na₂O₈P.

25. The chemotherapy agent of claim 24, wherein the cancer comprises at least one condition or disorder selected from the group consisting of esophageal cancer, prostate cancer, and breast cancer.

26. The chemotherapy agent of claim 24, wherein the non-cancerous condition comprises rheumatoid arthritis, pre-clampsia, or both.

27. The compound of claim 24, wherein the compound comprises a prodrug of an estradiol derivative.

28. The compound of claim 27, wherein the estradiol derivative comprises 2-methoxyestradiol (2-ME₂).

29. A metabolite comprising the compound of claim 24.

30. A metabolite of the compound of claim 24.

31. A composition comprising the compound of claim 15.

32. A pharmaceutical formulation or composition comprising the compound of claim 24.

33. A prodrug comprising 2-methoxyestradiol (2-ME₂) including a hydrophilic moiety at 3-position.

34. A prodrug comprising 2-methoxyestradiol (2-ME₂) including an ester moiety at 17-position.

35. A prodrug comprising 2-methoxyestradiol (2-ME₂) including a bioreversible hydrophilic moiety at 3-position and an ester moiety at 17-position.

36. The prodrug of claim 35, wherein the ester moiety comprises at least member selected from the group consisting of a straight chain molecule, a branched chain molecule, a cyclic molecule, and a combination thereof.

37. The prodrug of claim 36, wherein the molecule comprises an alkyl anhydride moiety.

38. A method of enhancing bio-efficacy and/or bioavailability of 2-methoxyestradiol (2-ME₂) in a living being, comprising the steps of:
a) providing a prodrug comprising 2-methoxyestradiol (2-ME₂) including a bioreversible hydrophilic moiety at 3-position and an ester moiety at 17-position;
b) administering the prodrug to the living being;
c) cleaving off the hydrophilic moiety from the 3-position presystemically and/or systemically; and
d) masking the 17-position during a first-pass through the intestinal epithelium and liver.

39. The method of claim 38, wherein step c) comprises cleaving off the hydrophilic moiety at or adjacent the intestinal epithelium.

40. The method of claim 38, wherein step d) comprises de-esterification of the ester moiety.

41. A method for prophylaxis or treatment of cancer, or a non-cancerous condition, comprising the steps of:
a) administering a predetermined dose of a medicinal agent to a living being in need thereof;
b) wherein the medicinal agent comprises a prodrug of 2-methoxyestradiol (2-ME₂).

42. The method of claim 41, wherein the prodrug comprises the following general structure:

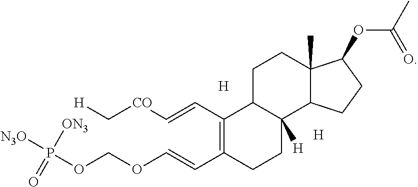

43. The method of claim 41, wherein the prodrug comprises the general formula C₂₂H₂₉Na₂O₈P.

44. The method of claim 41, wherein the prodrug comprises 2-methoxyestradiol (2-ME₂) including a bioreversible hydrophilic moiety at 3-position and an ester moiety at 17-position.

45. The method of claim 41, wherein the dose comprises about 10 mg/kg to about 31.5 mg/kg.

46. The method of claim 45, wherein the dose is administered orally or intravenously.

47. The method of claim 41, wherein the cancer comprises at least one condition or disorder selected from the group consisting of esophageal cancer, prostate cancer, and breast cancer.

48. The method of claim 41, wherein the non-cancerous condition comprises rheumatoid arthritis, pre-clampsia, or both.
